(19) United States
(12) Patent Application Publication
(10) Pub. No.: US 2024/0325712 A1
(43) Pub. Date: Oct. 3, 2024

(54) SURFACE TREATMENT FOR CONNECTORS

(71) Applicant: CareFusion 303, Inc., San Diego, CA (US)

(72) Inventor: Archana NAGARAJA RAO, Los Angeles, CA (US)

(21) Appl. No.: 18/616,678

(22) Filed: Mar. 26, 2024

Related U.S. Application Data
(60) Provisional application No. 63/454,887, filed on Mar. 27, 2023.

(51) Int. Cl. A61M 39/10

(52) U.S. Cl. CPC A61M 39/10; A61M 2205/02

(57) ABSTRACT

A connector for an infusion tubing has a hydrophobic and antistatic coating on an external surface thereof. The hydrophobic and antistatic coating is non-reactive to isopropyl alcohol and chlorhexidine. A method of treating a connector for an infusion line may include providing a coating of a hydrophobic and antistatic material that is non-reactive to isopropyl alcohol and chlorhexidine on an external surface of the connector.

### CROSS REFERENCE TO RELATED APPLICATION

This application claims the benefit of priority under 35 U.S.C. § 119 to U.S. Provisional Patent Application Ser. No. 63/454,887, entitled “SURFACE TREATMENT FOR CONNECTORS”, filed on Mar. 27, 2023, the entire contents of which is incorporated herein by reference.

### BACKGROUND

### Field

The present disclosure relates to the field of surface modification of polymeric materials. More specifically, the present disclosure relates to surface modification for improving chemical resistance of materials used for making connectors.

### Background

Tubing lines used for infusing therapeutics into patients often include connectors for connecting together various components of the tubing lines. Luer connectors are commonly used for such applications because of their reliability and ease of use. However, such connectors are also prone to failure because of various factors including exposure to reactive chemicals such as alcohol, interaction with infusates with a wide range of pH and reactivity, and mechanical stress from connections with other connector parts.

In particular, male luer parts tend to have higher failure rates because the external surface of the male luer part is exposed to friction and may become reactive to residual chemicals because of change in surface chemistry. Plastic male luer components have to comply with certain ISO standards such as ISO 594-2 and 80369-7 for dimensional and functional compliance. Commonly used materials for connectors include, but are not limited to, ABS, rigid PVC, and acrylic polymers. However, considering the wide variety of chemicals and mechanical stresses to which connectors are exposed to, different materials or surface treatments may be needed to increase chemical resistance while maintaining the bulk properties of the connectors.

### SUMMARY

The techniques disclosed herein advantageously can provide improved reliability of connectors used for infusion tubing. Moreover, the techniques herein can increase chemical resistance of connectors used for infusion tubing. Advantageously, the techniques disclosed herein can also reduce mechanical failure, e.g., cracking, of connectors caused by chemical breakdown of the connector material without compromising the bulk mechanical and/or chemical properties of the connector material.

In addition, methods described herein relate to the degradation of surface properties of connectors caused by exposure to reactive chemicals and drug compositions as well as solvents and solutions having a wide range of pH values. In particular, those methods are formulated to improve chemical resistance of external surfaces of connectors used for connecting infusion tubing. In addition, disclosed methods provide inexpensive techniques for increasing chemical resistance of connectors and reduce failure events that can cause leaks, inconvenience to patients and caregivers, and wastage of drug compositions. Even though specific use examples include the medical industry, these solutions have wide application potentials in other industries.

In one embodiment, a connector for an infusion tubing is provided. The connector has a hydrophobic and antistatic coating on an external surface thereof. The hydrophobic and antistatic coating is non-reactive to isopropyl alcohol and chlorhexidine.

In another embodiment, a method of treating a connector for an infusion tubing may include providing the coating of a hydrophobic and antistatic material that is non-reactive to isopropyl alcohol and chlorhexidine on an external surface of the connector.

In another embodiment, a method is provided of testing chemical resistance of a connector for infusion tubing. The method may include measuring a contact angle with water on an external surface of the connector to obtain a first water contact angle WCA1. The connector is then immersed in a solution having isopropyl alcohol for a period of time ranging from about 1 hour to about 6 hours. The contact angle with water on the external surface of the connector is then measured after removing the connector from the solution having isopropyl alcohol to obtain a second water contact angle WCA2. WCA1 and WCA2 are then compared and if WCA2 is within about 10% of WCA1, the connector is deemed to have acceptable chemical resistance.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 shows a Luer-Tubing Assembly.

FIGS. 2A-2D show a male Luer hub in various connector configurations.

FIGS. 3 and 4 show cross-section of a male Luer connector.

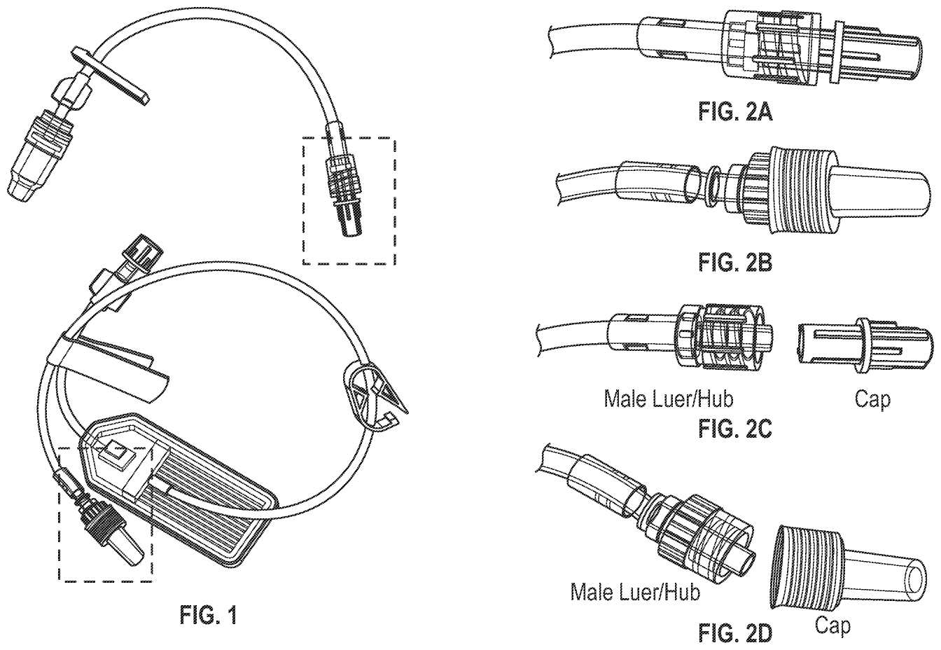

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for case of understanding.

Typical materials used for making connectors for infusion tubing (e.g., in medical or laboratory applications) include, but are not limited to polyvinyl chloride, acrylonitrile butadiene styrene (ABS), polycarbonate, an acrylic polymer, and a thermoplastic alloy. These materials are selected because of their rigidity and flexural modulus. However, the materials also tend to be reactive to disinfecting materials such as alcohol or chlorhexidine. Failure of connectors used in infusion tubing can be caused by several factors including, but not limited to, reaction with chemicals in disinfecting materials such as, for example, alcohol or chlorhexidine; reaction with chemicals being infused through the tubing which may include infusates with a wide range of pH, organic and inorganic solvents, lipids, etc.; and mechanical stress when connecting and disconnecting the connectors. Such failures of the connectors lead to leaks and loss of medical and patient inconvenience.

Male luer connectors are particularly prone to cracks due to alcohol or other disinfecting chemicals (e.g., chlorhexidine) when the connectors are disinfected using, for example disinfectant wipes to maintain sterility. The alcohol can cause damage to the surface of the male luer connector by cracking the male luer connector as it reacts with the material of the luer connector. Additionally, engagement with a female luer or other NAC can cause it to crack and fail because of the mechanical stress on the male luer connector during connection and disconnection.

In order to avoid such failure, one potential solution is to coat the external surface of the connectors with a hydrophobic and/or antistatic coating which is non-reactive to alcohol and other disinfectants. Some factors in determining the material for such coatings, without limitation, are: (a) ability to provide coatings of thicknesses ranging from a few nanometers to few microns; (b) hydrophobic or antistatic; (c) durable coating; and (d) non-reactive to disinfecting materials such as alcohol and chlorhexidine.

Accordingly, in an aspect of the present disclosure, a coating is provided for coating an external surface of connector. In some embodiments, the coating may include a hydrophobic material such as silicone and/or other highly hydrophobic material. Hydrophobic coating/super hydrophobic coating such as silicone can repel water, dirt, infusates with the hydrophilic nature, and are generally inert to disinfectants such as alcohol and/or chlorhexidine. The hydrophobic surface can create a higher contact angle that rolls of any solution in contact with the external surface of the connector thereby causing less of interaction between the fluid and the material of the connector. Because the residence time of contact of fluid on the material is reduced, the chemical reaction between the bulk material of the connector and the infusate/disinfecting wipes or the any of the drugs that might be infused to the patient is prevented or avoided. Thus, in some embodiments, the coating is inert or non-reactive to solvents and/or chemicals used in drugs or drug formulations such as, for example, organic solvents, lipids, solutions having a wide range of pH, etc., that are to be infused through the tubing and the connectors.

In some embodiments, the coating may be an organosilicate coating. Examples of organosilicates include, but are not limited to, polyorganosiloxanes such as polydimethylsiloxane (PDMS), polymethylphenylsiloxane (PMPS); polyurethane organosilicate (PUNC), etc. The thickness of the coating may range from about 10 nm to about 1 μm. For example, in some embodiments, the thickness of the coating may be about 10 nm, about 20 nm, about 30 nm, about 40 nm, about 50 nm, about 60 nm, about 70 nm, about 80 nm, about 90 nm, about 100 nm, about 120 nm, about 140 nm, about 160 nm, about 180 nm, about 200 nm, about 220 nm, about 240 nm, about 260 nm, about 280 nm, about 300 nm, about 320 nm, about 340 nm, about 360 nm, about 380 nm, about 400 nm, about 420 nm, about 440 nm, about 460 nm, about 480 nm, about 500 nm, about 525 nm, about 550 nm, about 575 nm, about 600 nm, about 625 nm, about 650 nm, about 675 nm, about 700 nm, about 750 nm, about 800 nm, about 850 nm, about 900 nm, about 950 nm, about 1000 nm, or any other thickness between any two of these thicknesses.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to 5 percent of the actual value stated, and other suitable tolerances.

The coatings are configured to render the surface of the connectors hydrophobic. Consequently, a contact angle of a drop of water on the coated surface is greater than 90°. In some embodiments, the contact angle is at least about 95°, at least about 97º, at least about 100°, at least about 102°, at least about 105°, at least about 107º, at least about 110°, at least about 115°, at least about 120°, at least about 125°, at least about 130°, or at least about 135°. In some embodiments, the coating results in an increase in the contact angle of a drop of water on an uncoated surface.

In some embodiments, the connector may be any connector used for connecting different components used for medical or laboratory applications. For example, in some embodiments, the connector may be any connector used for connecting two tube lines, or a tube line to a syringe, a reservoir bag or a cap. In some embodiments, the connector is a luer connector. In some embodiments, the connector is a male luer connector.

According to another aspect of the present disclosure, a method of providing a coating on a connector is provided. The method may include providing a coating of a hydrophobic and antistatic material that is non-reactive to isopropyl alcohol and chlorhexidine on an external surface of the connector. In some embodiments, the coating is an organosilicate.

In some embodiments, providing the coating involves coating the external surface of the connector using a plasma enhanced chemical vapor deposition (PE-CVD) process. In some embodiments, precursors used in the PE-CVD process may include, but are not limited to, tetraethoxysiline, hexamethyldisiloxane, methylcyclosiloxane, hexamethylcyclotrisiloxane, octamethylcyclotetrasiloxane, or a combination thereof. The PE-CVD process may be conducted in a low-pressure plasma reactor, with magnetrons which provide pulsed microwave radiation for generation of plasma. One of ordinary skill in the art will appreciate that parameters such as power, frequency, pulse width, and peak pulse power associated with the microwave radiation are dependent on the particular material being used including the precursors as well as the material of the connector. However, one of ordinary skill in the art will be able to suitably tailor the process for the particular materials being used.

In some embodiments, providing the coating involves dipping the connector into a liquid including the hydrophobic and antistatic material (e.g., an organosilicate) of interest. In such embodiments, the process may be conducted in an inert environment such as a chamber filled with an inert gas such as argon or nitrogen. In some embodiments, the liquid may include a suitable organosilicate precursor such as a polymethylsiloxane or a polyphenylsiloxane in a suitable solvent including toluene or xylene. Following the dipping of the connector into the liquid, the connector may be removed from the liquid and dried for a suitable amount of time to remove all the solvent, leaving behind a coating of the organosilicate on the external surface of the connector.

In some embodiments, providing the coating involves spraying a suitable aerosol including the hydrophobic and antistatic material of interest on to the connector. A liquid such as one used for dip-coating may be aerosolized and sprayed on to the connector surfaces. Those of ordinary skill in the art would appreciate the advantages and disadvantages of spray coating process relative to a dip-coating process.

In some embodiments, the process is configured to result in a coating having a thickness ranging from about 10 nm to about 1 μm. For example, in some embodiments, the thickness of the coating may be about 10 nm, about 20 nm, about 30 nm, about 40 nm, about 50 nm, about 60 nm, about 70 nm, about 80 nm, about 90 nm, about 100 nm, about 120 nm, about 140 nm, about 160 nm, about 180 nm, about 200 nm, about 220 nm, about 240 nm, about 260 nm, about 280 nm, about 300 nm, about 320 nm, about 340 nm, about 360 nm, about 380 nm, about 400 nm, about 420 nm, about 440 nm, about 460 nm, about 480 nm, about 500 nm, about 525 nm, about 550 nm, about 575 nm, about 600 nm, about 625 nm, about 650 nm, about 675 nm, about 700 nm, about 750 nm, about 800 nm, about 850 nm, about 900 nm, about 950 nm, about 1000 nm, or any other thickness between any two of these thicknesses.

Thus, in some embodiments, the various parameters of the process (PE-CVD, dip-coating or spray coating) may be tailored to obtain the desired thickness. For example, in a PE-CVD process, the infusion rate or volume of precursors introduced into the reaction chamber may be increased to provide a thicker coating.

In some embodiments, the process may include pretreatment of the external surface of the connector before providing the coating. For example, the connector surface may be exposed to plasma (e.g., oxygen plasma) to clean and/or activate the surface of the connector prior to applying the coating (e.g., via a PE-CVD process).

### EXAMPLES

### Example 1: Water Contact Angle

Water contact angle for connectors with coated and uncoated surfaces were measured. The following sets of connectors were tested: Set 1—untreated connectors; Set 2—connectors coated with organosilicate film (215 nm); Set 3—connectors coated with organosilicate film (152 nm) after pretreating the connectors with a plasma; and Set 4—connectors coated with organosilicate film (360 nm) after pretreating the connectors with a plasma and using a high precursor flow during the coating process.

The results of the test are summarized in Table 1 below:

TABLE 1

| | Connector | Contact | Film |
|---|---|---|---|
| Set | Type (Luer) | Angle | Thickness (nm) |
| 1 | Borla | 89 | |
| 1 | Medegen | 102 | |
| 2 | Borla | 106 | 215 |
| 2 | Medegen | 123 | |
| 3 | Borla | 100 | 152 |
| 3 | Medegen | 110 | |
| 4 | Borla | 99 | 360 |
| 4 | Medegen | 134 | |

### Example 2: Chemical Resistance Analysis

For testing chemical resistance, water contact angle for various coated connectors was measured after exposing the coated connectors to disinfectant wipes and isopropyl alcohol. For the first test, the coated connectors were wiped using chlorhexidine gluconate wipes for about 15 seconds followed by drying for about 30 seconds. Water contact angle was measured after drying. The results are summarized in Table 2 below:

TABLE 2

| | | Contact Angle |
|---|---|---|
| | Connector | (after disin- |
| Set | Type (Luer) | fectant wipe) |
| 2 | Borla | 77 |
| 2 | Medegen | 52 |
| 3 | Borla | 66 |
| 3 | Medegen | 65 |
| 4 | Borla | 45 |
| 4 | Medegen | 43 |

For the second test, the coated connectors were soaked in isopropyl alcohol for about 4 hours and the allowed to dry in air. Water contact angle was measured after drying. The results are summarized in Table 3 below:

TABLE 3

| | | | Contact Angle |
|---|---|---|---|
| | Connector | Contact Angle | (after soaking |
| Set | Type (Luer) | (before) | in IPA) |
| 2 | Borla | 108 | 103 |
| 2 | Medegen | 109 | 98 |
| 3 | Borla | 94 | 103 |
| 3 | Medegen | 105 | 97 |
| 4 | Borla | 96 | 93 |
| 4 | Medegen | 102 | 93 |

It is postulated that the reduction in contact angle after the chlorhexidine gluconate wipe is because of the residual chlorhexidine gluconate left on the surface after the connectors are wiped and dried with the wipe. A visible yellowish coating was visible on the surface of coated and uncoated connectors that were wiped with the chlorhexidine gluconate wipes.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

Clause 1. A connector for an infusion line, having a hydrophobic and antistatic material coated on an external surface thereof, the hydrophobic and antistatic material being non-reactive to isopropyl alcohol and chlorhexidine.

Clause 2. The connector of clause 1, wherein the hydrophobic and antistatic material comprises an organosilicate.

Clause 3. The connector of any of clauses 1-2, wherein the hydrophobic and antistatic material has a thickness in a range from 10 nm to 1 μm.

Clause 4. The connector of any of clauses 1-3, wherein the connector comprises a polymer material selected from the group consisting of polyvinyl chloride, acrylonitrile butadiene styrene (ABS), polycarbonate, an acrylic polymer, and a thermoplastic alloy.

Clause 5. The connector of any of clauses 1-4, wherein the connector has a flexural modulus of at least about 700 MPa.

Clause 6. The connector of any of clauses 1-5, wherein the hydrophobic and antistatic material is selected to have a water contact angle of at least 95°.

Clause 7. The connector of clause 6, wherein the water contact angle is not reduced by more than 10% after interaction with isopropyl alcohol.

Clause 8. The connector of any of clauses 1-7, wherein the connector is a luer connector.

Clause 9. The connector of clause 8, wherein the connector is a male luer connector.

Clause 10. A method of treating a connector for an infusion line, the method comprising: providing a coating of a hydrophobic and antistatic material that is non-reactive to isopropyl alcohol and chlorhexidine on an external surface of the connector.

Clause 11. The method of clause 10, wherein providing the coating comprises applying the hydrophobic and antistatic material using a plasma enhanced chemical vapor deposition (PECVD) process.

Clause 12. The method of any of clauses 10-11, wherein providing the coating comprises applying the hydrophobic and antistatic material by dipping the connector in a liquid comprising the hydrophobic and antistatic material.

Clause 13. The method of any of clauses 10-12, wherein providing the coating comprises applying the hydrophobic and antistatic material by spraying an aerosol comprising the hydrophobic and antistatic material.

Clause 14. The method of any of clauses 10-13, wherein the hydrophobic and antistatic material comprises an organosilicate material.

Clause 15. The method of any of clauses 10-14, wherein the connector comprises a polymer material selected from the group consisting of polyvinyl chloride, acrylonitrile butadiene styrene (ABS), polycarbonate, an acrylic polymer, and a thermoplastic alloy.

Clause 16. The method of any of clauses 10-15, wherein the connector is a luer connector.

Clause 17. The method of any of clauses 10-16, wherein providing the coating comprises applying a coating of the hydrophobic and antistatic material having a thickness in a range from 10 nm to 1 μm.

Clause 18. The method of any of clauses 10-17, further comprises pretreatment of the external surface of the connector prior to providing the coating.

Clause 19. The method of clause 18, wherein the pretreatment comprises exposing the external surface to a plasma.

Clause 20. A method for testing chemical resistance of a connector for infusion tubing, the method comprising: measuring a contact angle with water on an external surface of the connector to obtain a first water contact angle WCA1; immersing the connector in a solution comprising isopropyl alcohol for a period ranging from about 1 hour to about 6 hours; measuring the contact angle with water on the external surface of the connector after removing the connector from the solution comprising isopropyl alcohol to obtain a second water contact angle WCA2; comparing WCA1 and WCA2; and determining that the connector has acceptable chemical resistance if WCA2 is within about 10% of WCA1.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

In one or more aspects, the terms “about,” “substantially,” and “approximately” may provide an industry-accepted tolerance for their corresponding terms and/or relativity between items, such as from less than one percent to five percent.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A connector for an infusion line, having a hydrophobic and antistatic material coated on an external surface thereof, the hydrophobic and antistatic material being non-reactive to isopropyl alcohol and chlorhexidine.

2. The connector of claim 1, wherein the hydrophobic and antistatic material comprises an organosilicate.

3. The connector of claim 1, wherein the hydrophobic and antistatic material has a thickness in a range from 10 nm to 1 μm.

4. The connector of claim 1, wherein the connector comprises a polymer material selected from the group consisting of polyvinyl chloride, acrylonitrile butadiene styrene (ABS), polycarbonate, an acrylic polymer, and a thermoplastic alloy.

5. The connector of claim 1, wherein the connector has a flexural modulus of at least about 700 MPa.

6. The connector of claim 1, wherein the hydrophobic and antistatic material is selected to have a water contact angle of at least 95°.

7. The connector of claim 6, wherein the water contact angle is not reduced by more than 10% after interaction with isopropyl alcohol.

8. The connector of claim 1, wherein the connector is a luer connector.

9. The connector of claim 8, wherein the connector is a male luer connector.

10. A method of treating a connector for an infusion line, the method comprising:
providing a coating of a hydrophobic and antistatic material that is non-reactive to isopropyl alcohol and chlorhexidine on an external surface of the connector.

11. The method of claim 10, wherein providing the coating comprises applying the hydrophobic and antistatic material using a plasma enhanced chemical vapor deposition (PECVD) process.

12. The method of claim 10, wherein providing the coating comprises applying the hydrophobic and antistatic material by dipping the connector in a liquid comprising the hydrophobic and antistatic material.

13. The method of claim 10, wherein providing the coating comprises applying the hydrophobic and antistatic material by spraying an aerosol comprising the hydrophobic and antistatic material.

14. The method of claim 10, wherein the hydrophobic and antistatic material comprises an organosilicate material.

15. The method of claim 10, wherein the connector comprises a polymer material selected from the group consisting of polyvinyl chloride, acrylonitrile butadiene styrene (ABS), polycarbonate, an acrylic polymer, and a thermoplastic alloy.

16. The method of claim 10, wherein the connector is a luer connector.

17. The method of claim 10, wherein providing the coating comprises applying a coating of the hydrophobic and antistatic material having a thickness in a range from 10 nm to 1 μm.

18. The method of claim 10, further comprises pretreatment of the external surface of the connector prior to providing the coating.

19. The method of claim 18, wherein the pretreatment comprises exposing the external surface to a plasma.

20. A method for testing chemical resistance of a connector for infusion tubing, the method comprising:
measuring a contact angle with water on an external surface of the connector to obtain a first water contact angle WCA1;
immersing the connector in a solution comprising isopropyl alcohol for a period ranging from about 1 hour to about 6 hours;
measuring the contact angle with water on the external surface of the connector after removing the connector from the solution comprising isopropyl alcohol to obtain a second water contact angle WCA2;
comparing WCA1 and WCA2; and
determining that the connector has acceptable chemical resistance if WCA2 is within about 10% of WCA1.
